(19) United States
(12) Patent Application Publication
(10) Pub. No.: US 2026/0315029 A1
(43) Pub. Date: Oct. 15, 2026

(54) METHODS AND SYSTEMS FOR ULTRASOUND TOMOGRAPHY ANGLE-INDEPENDENT VECTOR BLOOD FLOW MEASUREMENT

(71) Applicant: UNIVERSITY OF ROCHESTER, Rochester, NY (US)

(72) Inventors: Mohammad MEHRMOHAMMADI, Pittsford, NY (US); Gaofei JIN, Rochester, NY (US)

(21) Appl. No.: 19/463,912

(22) Filed: Jan. 29, 2026

Related U.S. Application Data
(60) Provisional application No. 63/758,800, filed on Feb. 14, 2025.

(51) Int. Cl. A61B 8/13; A61B 8/00; A61B 8/06

(52) U.S. Cl. CPC A61B 8/13; A61B 8/06; A61B 8/4461; A61B 8/4483; A61B 8/466; A61B 8/5207; A61B 8/5276

(57) ABSTRACT

Enhanced ultrasound tomography vector flow imaging system and method use an enclosed or partially enclosed ring-array transducer and tomographic imaging to capture Doppler frequency shifts from multiple directions. This multi-angle detection improves flow vector reconstruction by solving an overdetermined reconstruction problem. The method and system significantly enhance accuracy and performance, offering a solution for precise, angle-independent blood flow imaging. This advancement improves disease diagnosis and treatment monitoring by providing more detailed and reliable vascular flow data.

This application claims priority of U.S. Provisional Patent Application No. 63/758,800, filed Feb. 14, 2025, the entirety of which is incorporated herein by reference.

### FIELD

This application relates generally to the field of ultrasound imaging, and in particular to ultrasound imaging and measurement of blood flow.

### BACKGROUND

Noninvasively visualizing blood flow in deep tissues provides critical insights for diagnosing diseases characterized by abnormal flow patterns, such as atherosclerosis, portal hypertension, and aneurysms. Imaging techniques like X-ray, CT, and MRI are commonly used but require contrast agents. In contrast, ultrasound, which relies on the Doppler effect, can provide real-time blood flow imaging without the need for such agents. However, conventional color Doppler ultrasound only measures flow along the direction of the ultrasound beam (axial flow), making it challenging to fully interpret complex flow dynamics and requiring significant expertise. There still exists a need for improved ultrasound methods and systems for real-time blood flow measurements.

### SUMMARY

One aspect of the application relates to a method of ultrasound tomography angle-independent vector blood flow measurement. The method comprises the steps of (1) acquiring data using an ultrasound transducer array with extended aperture size, wherein the extended aperture size is reflected in a relative ratio between the array aperture size and a largest dimension of an image object, wherein diverging waves are transmitted by a portion of the array using one or more elements of the array, wherein a divergent wavefront provides a field-of-view comprising anisotropic Doppler interrogation angles, wherein, after transmission of the diverging waves, the extended aperture of the array captures at least a majority of echo reflection waves, wherein the data is acquired by (a) rotating active views around the image object, wherein signal transmissions and echo receptions are repeated at each active view position, wherein a Doppler shift is acquired from multiple directions; and (b) distributing tomographic scanning sparsely and repeating at an ultrafast pulse repetition rate (PRF) (kHZ), wherein in one round of tomographic scanning the date acquired from a total number of M views are collected and the achievable frame rate is PRF/M; (2) dividing the portion of the ring array receiving the echoes at each view into N sub-apertures, wherein the echoes from each sub-aperture are beamformed in parallel at each time point, and a slow-time relative to the PRF ensemble of beamformed reflection images are saved, and estimating color Doppler shifts over the ensembled images; (3) collecting total MN Doppler measurements with tomographic scanning and sub-aperture beamforming techniques; (4) constructing a forward matrix A, wherein for the color Doppler measurements only the frequency shifts along the wave propagation direction are measured to produce a measurement matrix B, wherein a transmitted Doppler direction is pointing from pixels to the virtual point source of the diverging waves, and wherein a received Doppler direction is pointing from pixels to the virtual received source at the center of each sub-aperture; (5) reconstructing, after completion of the forward matrix A and measurement matrix B, the axial and lateral components of flow vectors by a least-squares solver; and (6) displaying an image for the image object.

Another aspect of the application relates to a method for three-dimensional ultrasound tomography vector flow imaging (3D UST-VFI). The method comprises the steps of (1) acquiring ultrasound data for UST-VFI processing; (2) estimating in-plane velocity components; (3) defining a region of interest for decorrelation analysis; (4) computing temporal correlation as an exponential function of time lag; (5) estimate a decorrelation value from the correlation function; (6) relating the decorrelation value to magnitudes of 3D motion; (7) estimate the out-of-plane velocity component using the known in-plane components; and (8) output a three-dimensional velocity estimate and/or derived flow metrics.

Another aspect of the application relates to a system for ultrasound tomography angle-independent vector blood flow measurement. The system comprises (1) an ultrasound transducer array with extended aperture size, wherein the extended aperture size is reflected in a relative ratio between the array aperture size and a largest dimension of an imaged object, wherein diverging waves are transmitted by a portion of the array using one or more elements of the array, wherein a divergent wavefront provides a field-of-view comprising anisotropic Doppler interrogation angles, wherein, after transmission of the diverging waves, the extended aperture of the ring array captures at least a majority of echo reflection waves; (2) a rotational mechanism connected to the ultrasound transducer array, wherein the rotational mechanism rotates the ultrasound transducer array through active views around the imaged object; (3) a computer processor, wherein the computer processor (a) estimates color Doppler shifts over the ensembled images, (b) constructs a forward matrix A, wherein for the color Doppler measurements only the frequency shifts along the wave propagation direction are measured to produce a measurement matrix B, wherein a transmitted Doppler direction is pointing from pixels to the virtual point source of the diverging waves, and wherein a received Doppler direction is pointing from pixels to the virtual received source at the center of each sub-aperture, and (c) reconstructs, after completion of a forward matrix A and measurement matrix B, the axial and lateral components of flow vectors by a least-squares solver; and (4) a display device connected to the computer processor.

### BRIEF DESCRIPTION OF THE DRAWINGS

Some aspects of the claimed methodology are illustrated but not limited to the figures in the accompanying drawings. In particular, while certain specific transducer arrays are shown in many figures (e.g., enclosed ring or concaved/partial ring array transducer), these are for demonstration and the methodology is applicable to any transducer array with extended aperture size.

FIGS. 1A and 1B show illustration of ultrasound tomography-vector flow imaging (UST-VFI). In FIG. 1A, tomographic imaging is achieved by electrically rotating the transmit and receive elements. At each view, a single element (light blue) transmits diverging waves, while the neighboring elements (dark blue) receive the echoes. As shown in FIG. 1B, the receive aperture is split into n sub-apertures, within which the received echoes are beamformed in parallel and Doppler frequency shifts are estimated from all frames in each sub-aperture over slow time.

FIG. 2 shows an example of some typical configurations of ultrasound array with extended aperture, including enclosed and partially enclosed cases.

FIGS. 3A-3F show comparison of the estimated lateral (FIGS. 3A-3C) and axial (FIGS. 3D-3F) velocity components for all views (FIGS. 3B, 3E) and a single view (FIGS. 3C, 3F) with the ground truth velocity maps (FIGS. 3A, 3D). Histogram of the reconstructed errors and the fitted Student's t-distribution for all views (FIG. 3H) and a single view (FIG. 3I). The coefficient of determination for all fits is greater than 0.9.

FIGS. 4A-4C show a comparison of the estimated magnitude maps for all views (FIG. 4B) and a single view (FIG. 4C) with the ground truth (FIG. 4A). FIGS. 4D-4E show reconstructed magnitude error maps for all views (FIG. 4D) and a single view (FIG. 4E). FIG. 4F shows the angular deviation of the error map along the radial distance from the phantom center.

FIGS. 5A-5C show a comparison of the estimated phase maps for all views (FIG. 5B) and a single view (FIG. 5C) with the ground truth (FIG. 5A). FIGS. 5D-5E show reconstructed phase error maps for all views (FIG. 5D) and a single view (FIG. 5E). FIG. 5F shows the angular deviation of the error map along the radial distance from the phantom center.

FIG. 6 shows a demonstration of how the extended view coverage influences the reconstruction accuracy. Reconstruction error analysis at different view coverage.

FIG. 7 shows an example of vector flow imaging using wall-less flow phantom, demonstrating the feasibility of in-vivo blood flow imaging. The reconstructed magnitude of flow vectors is comparable to the simulation results.

FIGS. 8A-8F show simulation results for out-of-plane flow estimation using speckle decorrelation. (FIG. 8A) Relative position and orientation of a straight tube phantom with respect to the transducer probe. (FIG. 8B) Enlarged view of the simulated laminar flow distribution within the tube phantom. (FIG. 8C) Gaussian fitting of the measured speckle correlation function used to estimate the decorrelation value. (FIG. 8D) Ground-truth out-of-plane velocity distribution. (FIG. 8E) Estimated out-of-plane velocity obtained using the speckle decorrelation method. (FIG. 8F) Reconstructed three-dimensional blood flow map combining in-plane and out-of-plane velocity components.

### DETAILED DESCRIPTION

Reference will be made in detail to certain aspects and exemplary embodiments of the application, illustrating examples in the accompanying structures and figures. The aspects of the application will be described in conjunction with the exemplary embodiments, including methods, materials and examples, such description is non-limiting and the scope of the application is intended to encompass all equivalents, alternatives, and modifications, either generally known, or incorporated here. Unless otherwise defined, all technical and scientific terms used herein have the same meaning as commonly understood by one of ordinary skill in the art to which this application belongs. One of skill in the art will recognize many techniques and materials similar or equivalent to those described here, which could be used in the practice of the aspects and embodiments of the present application. The described aspects and embodiments of the application are not limited to the methods and materials described.

### I. Definitions

As used in this specification and the appended claims, the singular forms “a”, “an” and “the” include plural referents unless the content clearly dictates otherwise.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about”, it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to the value”, “greater than or equal to the value” and possible ranges between values are also disclosed, as appropriately understood by the skilled artisan.

The term “ultrasound transducer array,” as used herein, refers to an ultrasound probe that contains multiple tiny piezoelectric crystals (elements) arranged in patterns (linear, phased, curvilinear, matrix) that can be pulsed individually or in sequences to electronically steer, focus, and create ultrasound beams, generating detailed images by sending sound waves into the body and interpreting the returning echoes. These arrays allow for versatile beam control, offering wide views, deep penetration, or high resolution depending on their design. The array elements may have different sizes, forms, and divisions for different bands.

### II. Overview

Vector Flow Imaging (VFI) is a techniques to reconstruct both lateral and axial components of blood flow. This is achieved through techniques such as transverse oscillation, multi-angle Doppler acquisition, or tracking moving speckle patterns. However, traditional VFI systems are limited by the small aperture size of linear and phased-array transducers, which restricts the detection of Doppler shifts to a narrow angle range, leading to inaccuracies, especially for lateral flow estimation.

Ultrasound tomography (UST) uses an enclosed ring-array transducer that surrounds the target area, enabling it to capture reflection, scattering, and transmitted waves without operator dependence. UST allows for the quantitative reconstruction of tissue properties such as sound speed, attenuation, and density, enhancing applications like breast cancer screening. Despite these benefits, current UST systems do not support blood flow imaging due to the complexity of acquiring and reconstructing Doppler information from multiple views.

This present application describes methods and systems that quantitatively measure blood flow measurements based on the vector flow imaging (VFI) principle. Conventional Doppler ultrasound faces a significant limitation in that it can only measure blood flow along the axis of the ultrasound beam, making it highly dependent on the vessel's angle relative to the transducer. This angular dependency often leads to errors in estimating flow dynamics and requires operators to position the transducer precisely to obtain accurate results. The method and the system of the present application address this limitation by using a transducer array that either fully or partially surrounds the target area, allowing for the detection of Doppler frequency shifts from multiple directions. By capturing flow information from various angles, the method and system of the present application reconstruct both axial and lateral velocity components to generate a comprehensive vector flow map. This results in a 2D and 3D visualization of blood flow, providing a much more accurate and intuitive representation of vascular dynamics. The ability to perform measurements independent of the vessel angle reduces the need for specialized operator expertise, making the method the and the system of the present application more user-friendly and less prone to human error. Additionally, the method and system of the present application support noninvasive imaging of deep tissue vasculature, which can improve the diagnosis and monitoring of diseases characterized by abnormal blood flow, such as atherosclerosis and aneurysms.

The method and system of the present application offer significant potential for clinical applications by enhancing the reliability of blood flow imaging. With its operator-independent design and full-field flow visualization capabilities, the method and system of the present application can serve as a powerful tool for improving diagnostic accuracy and monitoring treatment responses in various medical conditions. By overcoming the limitations of traditional Doppler ultrasound, the method and system of the present application provide a robust platform for real-time, quantitative assessment of tissue/vascular health.

### III. Method for Ultrasound Tomography Angle-Independent Vector Blood Flow Measurement

One aspect of the present application relates to a method for ultrasound tomography angle-independent vector blood flow measurement. The method comprises the steps of: (1) acquiring image data of an image object using an ultrasound transducer array with extended aperture size, (2) dividing the portion of ultrasound transducer array receiving echoes at each view into N sub-apertures, and estimating color Doppler shifts over the ensembled images, (3) collecting total MN Doppler measurements with tomographic scanning and sub-aperture beamforming techniques, (4) constructing a forward matrix A, wherein for the color Doppler measurements only the frequency shifts along the wave propagation direction are measured to produce a measurement matrix B, (5) reconstructing, after completion of the forward matrix A and the measurement matrix B, the axial and lateral components of flow vectors by a least-squares solver, and (6) displaying an image for the image object.

In some embodiments, the extended aperture size of the ultrasound transducer array is reflected in a relative ratio between the array aperture size and a largest dimension of an imaged object of >0.5 wherein in step (1) diverging waves are transmitted by a portion of the array using one or more elements of the array, wherein a divergent wavefront provides a field-of-view comprising anisotropic Doppler interrogation angles, wherein, after transmission of the diverging waves, the extended aperture of the ring array captures at least a majority of echo reflection waves. Image data is generated by (a) rotating active views around the imaged object, wherein signal transmissions and echo receptions are repeated at each active view position and a Doppler shift is acquired from multiple directions, and (b) distributing tomographic scanning sparsely and repeating at an ultrafast pulse repetition rate (PRF) (kHz), wherein one round of tomographic scanning the date acquired from a total number of M views are collected and the achievable frame rate is PRF/M. In some embodiments, the sparsity and spatial distribution of tomographic scanning in step (b) changes to accommodate different coverage and shape of the transducer array. In some embodiments, the aperture size is between about 15 mm and 75 mm, about 20 mm and 70 mm, about 25 mm and 65 mm, about 30 mm and 60 mm, or about 35 mm and 55 mm. In some embodiments, the aperture size is between about 38 mm and 50 mm. In some embodiments, the imaging depth is between about 1 cm and 20 cm, or about 5 cm and 15 cm. In some embodiments, the imaging depth is about 10 cm.

In some embodiments, the active views in step (1) are rotated around the imaged object electronically. In some embodiments, the active views in step (1) are rotated around the imaged object mechanically. In some embodiments, the Doppler shift in step (1) is acquired in a lateral direction relative to the imaged object.

In step (2), in some embodiments, echoes from each sub-aperture are beamformed in parallel at each time point, and a slow-time relative to the PRF ensemble of beamformed reflection images are saved. In some embodiments, clutter filtering is employed on the ensemble of reflection images after beamforming. In some embodiments, clutter filtering is employed on the ensemble of reflection images before beamforming. In some embodiments, estimation of color Doppler shifts uses a lag-one autocorrelator in a sliding window.

In step (4), in some embodiments, a transmitted Doppler direction is pointing from pixels to the virtual point source of the diverging waves, and a received Doppler direction is pointing from pixels to the virtual received source at the center of each sub-aperture.

In some embodiments, the ultrasound transducer array is a curved array with a degree of curvature in the range of 45°-360°, 45°-270°, 45°-180°, 45°-135°, 45°-90°, 90°-360°, 90°-270°, 90°-180°, 90°-135°, 135°-360°, 135°-270°, 135°-180°, 180°-360°, 180°-270°, or 270°-360°. In some embodiments, the curved array is a single continuously curved transducer array. In some embodiments, the curved array comprises two or more curved subarrays.

In some embodiments, the ultrasound transducer array is an enclosed ring array. In some embodiments, the ultrasound transducer array is a partially enclosed ring array. In some embodiments, the enclosed or partially enclosed ring array consists of a single continuously curved transducer array. In some embodiments, the enclosed or partially enclosed ring array consists of two or more curved subarrays.

In some embodiments, the image displayed in step (6) is a power Doppler imaging of the image object. In some embodiments, the image displayed in step (6) is of vector flow imaging of the image object.

### Data Acquisition

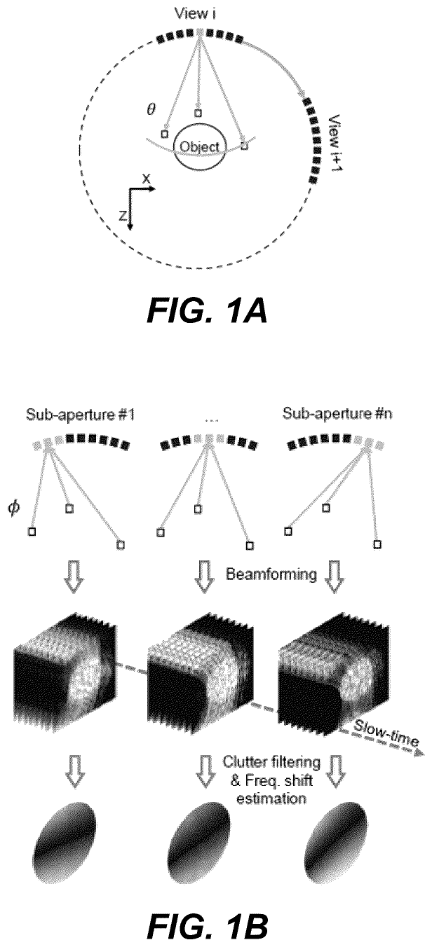

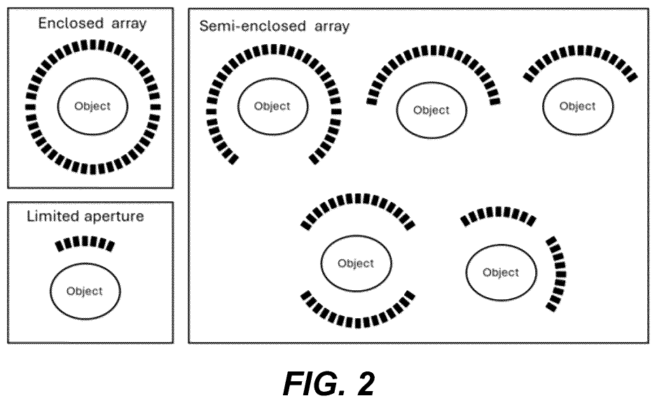

In some embodiments, an ultrasound transducer array with extended aperture size is used for data acquisition. Here, the definition of extended aperture refers to the relative ratio between the array aperture size and the largest dimension of the imaged object, typically including enclosed and partially enclosed cases (FIG. 2). Take the enclosed ring array as an example shown in FIG. 1A, in each tomographic view, diverging waves are transmitted by a portion of the ring array, using a single or multiple elements. This highly divergent wavefront provides a wide field-of-view and anisotropic Doppler interrogation angles. After transmission, the extended aperture of the ring array allows to capture all or most of the reflection waves to improve the reconstructed image quality. The active views are subsequently rotated around the imaged object by either electronically or mechanically and repeat both signal transmission and echo reception at each view. This tomographic scanning technique can acquire the Doppler shift from multiple directions, especially in the lateral direction which is not applicable for conventional VFI methods with limited aperture size. The tomographic scanning is distributed sparsely and repeated at ultrafast pulse repetition frequency or rate (PRF) (~kHz). The sparsity and spatial distribution of the tomographic views can change to accommodate different coverage and shape of the transducer array. It can also change depending on the region of interest where the VFI measurement is required. In one round of tomographic scanning, the data from total M views are collected. The achieved frame rate is PRF/M.

### Data Processing and Representation in Image Form

In some embodiments, the portion of the ring array used to receive the echoes at each view is divided into N sub-apertures (FIG. 1B). The echoes from each sub-aperture are beamformed in parallel at each time point, and a slow-time (relative to fast PRF) ensemble of beamformed reflection images are saved. To filter out the signals from stationary tissues and background noises, SVD-based clutter filtering is employed on the ensemble of reflection images. The clutter filtering may be conducted alternatively on the ensemble of echo signals before beamforming. Then, color Doppler shifts are estimated using a lag-one autocorrelator in a sliding window over the ensembled images. By reasonably selecting the window length and step size, a video of flow dynamics can be rendered after the flow vectors are reconstructed, which will be explained in the next step. Alternatively, different approaches for beamforming, clutter filtering and doppler shifts estimation may also be used.

In some embodiments, a total of M×N Doppler measurements are collected, wherein M is the number of view of tomographic scannings and N is the number of sub-apertures in beamforming techniques (FIG. 1B). The forward matrix A is constructed based on the principle of color Doppler measurements that only the frequency shifts along the wave propagation direction are measured. The transmitted Doppler direction is pointing from pixels to the virtual point source of the diverging waves, and the received Doppler direction is pointing from pixels to the virtual received source at the center of each sub-aperture. After completion of the forward matrix A and measurement matrix B, the axial and lateral components of flow vectors are reconstructed by a least-squares solver. Alternatively, different approaches for solving the inverse reconstruction problem and regularization strategies may also be used.

In some embodiments, the method further comprises the step of providing a diagnosis of based on the results of blood flow measurement of the image object. A number of conditions and diseases, for example, may be diagnosed based on the abnormal blood flow patterns, flow velocity, perfusion, or vascular remodeling, including but not limited to: oncologic applications, such as longitudinal evaluation of tumor vascularity and perfusion changes for assessing response to neoadjuvant or adjuvant chemotherapy, including breast cancer; vascular diseases, including abdominal aortic aneurysm (AAA) through assessment of aneurysm size, wall shear stress, thrombus formation, and flow asymmetry; peripheral and limb vascular disorders, such as deep vein thrombosis (DVT), peripheral arterial disease (PAD), venous insufficiency, and ischemia; cerebrovascular conditions, including carotid artery stenosis or thrombosis, plaque instability, and impaired cerebral perfusion; brain hemodynamic disorders, including ischemic stroke, hemorrhagic stroke, transient ischemic attack (TIA), cerebral hypoperfusion, and other neurovascular or neurodegenerative disorders; and functional brain assessment, including monitoring task- or stimulus-induced hemodynamic changes associated with neural activity for use in brain-computer interface (BCI) systems, neurofeedback, cognitive workload assessment, and functional brain mapping.

The method of the present application can also be used for detection or monitoring of pathological changes in a subject, such as breast tumor progression or tumor responses after treatment. The method of the present application can also be used for differentiation of benign from malignant masses.

Another aspect of the present application relates to a method for three-dimensional (3D) ultrasound tomography vector flow imaging (3D UST-VFI). The method comprises the steps of:
(1) acquiring ultrasound data for UST-VFI processing;
(2) estimating in-plane velocity components;
(3) defining a region of interest for decorrelation analysis;
(4) computing temporal correlation as an exponential function of time lag;
(5) estimate a decorrelation value from the correlation function;
(6) relating the decorrelation value to magnitudes of 3D motion;
(7) estimate the out-of-plane velocity component using the known in-plane components; and
(8) output a three-dimensional velocity estimate and/or derived flow metrics. In some embodiments,

In some embodiments, the in-plane velocity components in step (2) is estimated using the previously disclosed UST-VFI methods.

### IV. The Ultrasound Tomography Vector Flow Imaging (UST-VFI) System

Another aspect of the present application relates to an ultrasound tomography vector flow imaging (UST-VFI) system. The UST-VFI system comprises an ultrasound transducer array that is capable of generating tomography vector flow imaging with the method of the present application.

In some embodiments, the ultrasound tomography vector flow imaging (UST-VFI) system of the present application comprises an enclosed or partially enclosed ring-array ultrasound transducer with extended aperture size. In some embodiments, the enclosed or partially enclosed ring array consists of a single continuously curved transducer array. In some embodiments, the enclosed or partially enclosed ring array consists of two or more curved subarrays.

In some embodiments, the ultrasound tomography vector flow imaging (UST-VFI) system of the present application comprises a curved array with extended aperture size and a degree of curvature in the range of 45°-360°, 45°-270°, 45°-180°, 45°-135°, 45°-90°, 90°-360°, 90°-270°, 90°-180°, 90°-135°, 135°-360°, 135°-270°, 135°-180°, 180°-360°, 180°-270°, or 270°-360°. In some embodiments, the curved array is a single continuously curved transducer array. In some embodiments, the curved array comprises two or more curved subarrays.

In some embodiments, diverging waves are transmitted by a portion of the array using one or more elements of the array, wherein a divergent wavefront provides a field-of-view comprising anisotropic Doppler interrogation angles, wherein, after transmission of the diverging waves, the extended aperture of the ring array captures at least a majority of echo reflection waves.

In some embodiments, the system of the present application further comprises a rotational mechanism connected to the ultrasound transducer array, wherein the rotational mechanism rotates the ultrasound transducer array through active views around the imaged object.

In some embodiments, the system of the present application further comprises a computer processor. In some embodiments, the computer processor (a) estimates color Doppler shifts over the ensembled images, and constructs a forward matrix A, wherein for the color Doppler measurements only the frequency shifts along the wave propagation direction are measured to produce a measurement matrix B, wherein a transmitted Doppler direction is pointing from pixels to the virtual point source of the diverging waves, and wherein a received Doppler direction is pointing from pixels to the virtual received source at the center of each sub-aperture; and (b) reconstructs, after completion of a forward matrix A and measurement matrix B, the axial and lateral components of flow vectors by a least-squares solver.

In some embodiments, the system of the present application further comprises a display device, wherein the display device is connected to the computer processor, and is capable of displaying an image of the image object.

In conventional ultrasound (US) vector flow imaging (VFI) with linear array transducers, the limited aperture size restricts the detection of lateral Doppler frequency shifts, thereby limiting the capability of detecting flow omnidirectionally and the accuracy of vector flow reconstruction. In the system of the present application, the enclosed or partially enclosed ring-array or the curves array of ultrasound transducer is employed that fully or partially encloses the imaged object to interrogate frequency shifts from multiple angular directions. This UST-VFI approach enables accurate estimation of both axial and lateral components of flow vectors based on the detected Doppler frequency shifts in each tomographic view, using a least-squares estimator. As described in more details in the Examples, proof-of-concept in-vitro results from a rotary disk phantom demonstrated clear enhancements of UST-VFI in uniformity and a reduction in angle-dependent errors in both the magnitude and phase of the estimated flow vectors. By utilizing the estimated Doppler shifts from all tomographic views, the root-mean-square error (RMSE) was reduced by 49.2% in magnitude and 77.6% in phase estimation compared to the single-view scenario. These results show the omnidirectional detection capabilities of the UST-VFI of the present application and emphasize the role of expanding aperture size in achieving reliable vector flow reconstruction.

In some embodiments, the UST-VFI system of the present application has a data acquisition rate in the range of up to about 7 kHz. The UST-VFI system of the present application, with the ultrafast data acquisition rate, can provide both functional and quantitative imaging modalities. The maximum PRF is defined by Formula (1), where D is the diameter of the ring size and co is the sound speed.

$$PRF = \frac{c 0}{2 D} \qquad ( 1 )$$

### Measuring Out-of-Plane Velocity Component of Blood Flow

Another aspect of the present application relates to a method of . . .

In addition to the novel two-dimensional vector flow imaging described above, the present application provides a method and system for three-dimensional (3D) blood flow velocity vector measurement. A limitation of previous two-dimensional vector flow imaging methods is common to ultrasound imaging systems based on one-dimensional transducer arrays and planar image formation, in which motion orthogonal to the imaging plane is not directly observable using conventional in-plane velocity estimation techniques.

The present application addresses the foregoing limitation by introducing a speckle decorrelation-based approach for estimating the out-of-plane component of blood flow velocity. The disclosed approach exploits the observation that temporal decorrelation of ultrasound speckle is sensitive to scatterer motion in all spatial directions, including motion orthogonal to the imaging plane. By quantifying speckle decorrelation from beamformed ultrasound data and combining this information with the in-plane velocity components obtained using the previously disclosed UST-VFI methods, the out-of-plane velocity component can be estimated.

The presently disclosed combination of in-plane velocity estimation as described here and speckle decorrelation-based analysis as disclosed herein for out-of-plane velocity estimation enables estimation of three-dimensional blood flow velocity vectors using ultrasound data acquired with a two-dimensional imaging geometry. This therefore provides the advantage of extending the scope of the UST-VFI methods herein from two-dimensional vector flow imaging to three-dimensional blood flow measurement, without requiring additional hardware, mechanical scanning, or two-dimensional matrix transducer arrays.

The present application is further illustrated by the following examples that should not be construed as limiting. The contents of all references, patents, and published patent applications cited throughout this application, as well as the Figures and Tables, are incorporated herein by reference.

### EXAMPLES

### Example 1: UST-VFI Data Acquisition System and Experimental Setup

A sparse-view synthetic aperture sequence [J. A. Jensen, S. I. Nikolov, K. L. Gammelmark, and M. H. Pedersen, “Synthetic aperture ultrasound imaging,” Ultrasonics, vol. 44, pp. e5-e15, 2006 Dec. 22/2006] was implemented on a 256-channel ultrasound ring-array transducer with a 1.5 MHz central frequency for data acquisition. At each tomographic view, a single transducer element transmitted a diverging wavefield propagating through the imaged object, while a quarter of the ring array (64 elements) centered at the transmitting element received the echoes (FIG. 1A). A total of 8 evenly distributed views surrounding the object were fired sequentially at 3 kHz PRF using a tone burst signal of 2 half cycles, achieving a frame rate of 375 Hz over 40 mm radius. This high frame rate enables the UST system to detect the Doppler Nyquist velocity at 92.5 mm/s without frequency aliasing, as given by [D. H. Evans, Doppler ultrasound: Physics, instrumentation, and clinical applications. 1989]:

$${V}_{N} = \frac{cPRF}{4 {N}_{tx} {f}_{c}} \qquad ( 2 )$$

where c is the sound speed of the coupling water, Ntx is the number of sparse views in transmission, and fc represents the central frequency.

To obtain the ground truth (GT) of the vector flow profile in experiments, a disk phantom with a radius of 12 mm was driven by a rotary motor at an angular velocity ω of 1.67 rad/s (FIG. 1C). This configuration allowed a maximum velocity of 20 mm/s at the phantom edge, with the velocity magnitude profile as a function of radius given by V=ωr. Similarly, the phase map was determined based on the angle of each pixel inside the phantom relative to its center. The phantom was composed of 1.5% w/v agarose and 1% w/v cellulose power and molded in a 3D printed holder for stable rotation (FIG. 1C). To ensure continuous rotation during data acquisition, the rotary motor was individually controlled by driving software with a micro-stepping size of 0.036°. Data acquisition lasted approximately 2.67 s for a total of 1000 frames, during which the phantom completed 0.71 rotations.

### Vector Flow Reconstruction

To increase the number of measurements and detection directivity, the 64 received elements in each view were split into 4 non-overlapping sub-apertures (NsubApe=4, FIG. 1B). The echoes collected in each sub-aperture, comprising 16 elements, were beamformed in parallel, sharing the same PRF. After filtering the clutter [C. Demene et al., “Spatiotemporal Clutter Filtering of Ultrafast Ultrasound Data Highly Increases Doppler and fUltrasound Sensitivity,” IEEE Trans Med Imaging, vol. 34, no. 11, pp. 2271-85, November 2015], the color Doppler frequency shift was estimated using a classic lag-one autocorrelator [D. Evans, “Colour flow and motion imaging,” Proceedings of the Institution of Mechanical Engineers, Part H: Journal of Engineering in Medicine, vol. 224, no. 2, pp. 241-253, 2010] from the ensemble of sub-aperture beamformed images at each view (FIG. 1B). By electrically rotating the window of the active view (FIG. 1A), axial frequency shifts from all directions were successfully captured by the ring array, and the flow vector was reconstructed by solving the linear system AV=b [B. Y. Yiu and A. C. Yu, “Least-Squares Multi-Angle Doppler Estimators for Plane-Wave Vector Flow Imaging,” IEEE Trans Ultrason Ferroelectr Freq Control, vol. 63, no. 11, pp. 1733-1744 November 2016; I. K. H. Tsang, B. Y. S. Yiu, and A. C. H. Yu, “A least-squares vector flow estimator for synthetic aperture imaging,” in 2009 IEEE International Ultrasonics Symposium, 20-23 Sep. 2009 2009, pp. 1387-1390]:

$$[ \begin{matrix}cos {θ}_{1} + cos {ϕ}_{1} & sin {θ}_{1} + sin {ϕ}_{1} \\ ⋮ & ⋮ \\ cos {θ}_{i} + cos {ϕ}_{i} & sin {θ}_{i} + sin {ϕ}_{i} \\ ⋮ & ⋮ \\ cos {θ}_{M} + cos {ϕ}_{M} & sin {θ}_{M} + sin {ϕ}_{M}\end{matrix} ] [ \begin{matrix}{V}_{z} \\ {V}_{x}\end{matrix} ] = \frac{c}{{f}_{c}} [ \begin{matrix}Δ {f}_{1} \\ ⋮ \\ Δ {f}_{i} \\ ⋮ \\ Δ {f}_{M}\end{matrix} ] \qquad ( 3 )$$

The number of measurements is M=NₜₓN_{subApe}=32, which is substantially greater than the number of variables to be solved: the lateral velocity component (Vx) and axial velocity component (Vz) in this case. Here, θ and φ represent the interrogation angle in transmission and reception, respectively. The corresponding estimated axial frequency shift for the i-th measurement is denoted as Δfi. The least-squares solution of this overdetermined system is:

$$V = [ \begin{matrix}{V}_{z} \\ {V}_{x}\end{matrix} ] = {( {A}^{T} A )}^{- 1} {A}^{T} b \qquad ( 4 )$$

The error analysis was conducted in two groups, representing the fully tomographic VFI with the measurements from all views (Ntx=8, M=32) and the case of limited aperture size with measurements from only one view (Ntx=1, M=4). The estimation error was calculated relative to the ground truth, where positive values indicated overestimation, and negative values indicated underestimation. Since the true flow vector map of the rotary phantom was known, the discrepancy between the reconstructed lateral and axial error distributions in each group was analyzed using Kullback-Leibler (KL) divergence:

$${D}_{K L} ( P \vert \vert Q ) = {\sum }_{x ∈ ROI} P ( x ) log ( \frac{P ( x )}{Q ( x )} ) \qquad ( 5 )$$

Here, P and Q represent the error distributions of the estimated Vx and Vz, respectively. KL divergence quantifies the difference between the lateral and axial error distributions. After decoupling the flow vectors into lateral and axial components, both magnitude and phase were easily obtained, and their estimated errors were subsequently analyzed. To demonstrate the omnidirectional capability of UST-VFI, the uniformity of the error map was evaluated by calculating the standard deviation (STD) circumferentially at different radial distances.

Reduced Angular Dependency with UST-VFI

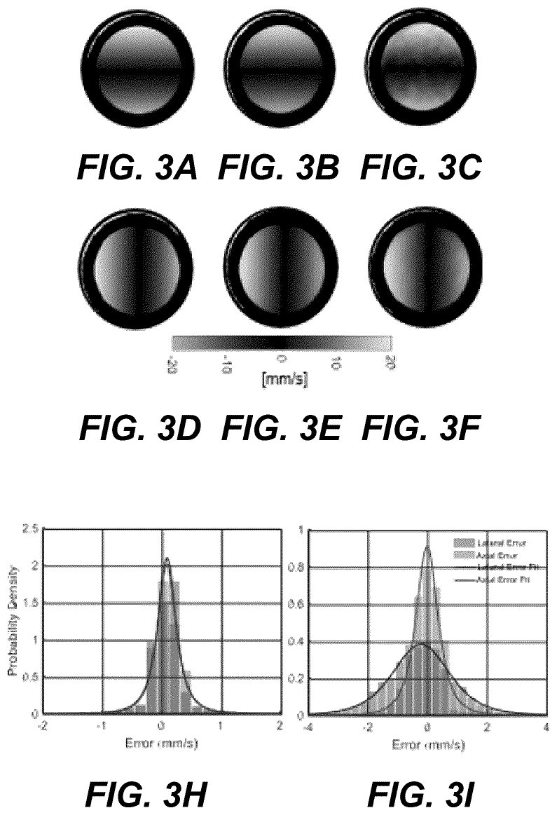

In the limited aperture scenario, where only a portion of the ring array captured the Doppler shifts, the range of interrogation angles was predominantly restricted to the axial direction. Although sub-aperture beamforming successfully isolated Doppler shifts in different directions on the reception side, enabling the estimation of lateral velocity (FIG. 3C), the reconstruction accuracy was lower in the lateral direction compared to the axial direction (FIG. 3F), as evaluated against the true velocity map (FIGS. 3A, 3D). By incorporating new interrogation angles from the lateral direction using a tomographic sequence, the variance of lateral velocity estimation decreased noticeably (FIG. 3B). This improvement was further supported by the probability density distribution of reconstruction errors: the STD of lateral errors decreased from 1.29 mm/s to 0.42 mm/s (FIGS. 3H & 3I, shown in blue) when comparing reconstructions using a single view to those using all tomographic views. Additionally, the KL divergence between the fitted lateral and axial error distributions reduced from 5.05 (FIG. 3I) to 0.70 (FIG. 3H) when all tomographic views were included, demonstrating greater similarity and reduced angle-dependent errors between the lateral and axial directions.

### Magnitude Estimation and Phase Estimation of Vector Flow of Vector

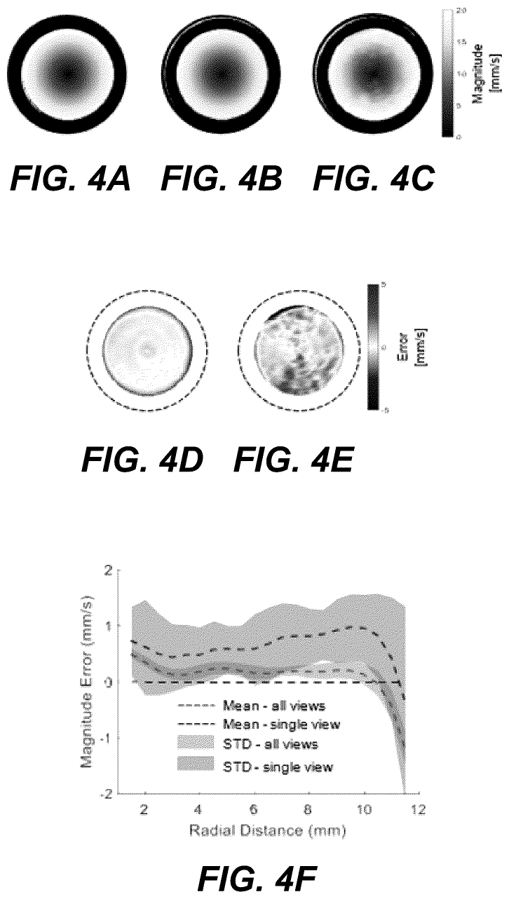

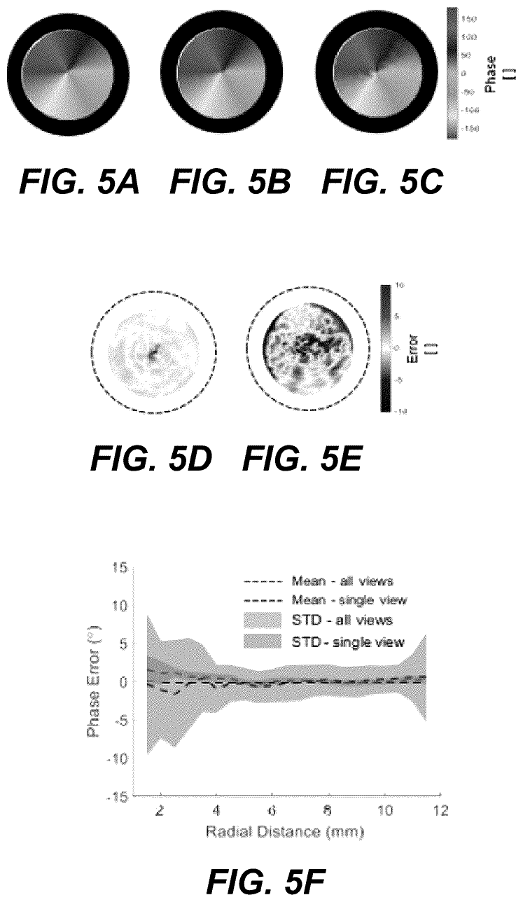

Demonstrating the accuracy improvements in VFI reconstruction using an extended ultrasound transducer array, a cylindrical phantom with a radius of 12 mm was rotated continuously by a rotary motor, resulting a linear increased velocity magnitude ranging from 0-20 mm/s and an omnidirectional flow pattern. The known velocity map of magnitude and phase allows to compare the reconstruction results quantitatively. In the presented drawings (FIGS. 4A-4F & FIGS. 5A-F), all-view and single-view case represent the invented UST-VFI with the enclosed ring-array and conventional VFI with the limited aperture size, respectively. With the introducing of new Doppler measurements from multiple directions surrounding the imaged object, UST-VFI is enabled to reconstruct more uniform flow vector map in terms of both magnitude and phase (FIGS. 4A-4E & FIGS. 5A-5E) and greatly reduce the estimation errors (FIG. 4F & FIG. 5F).

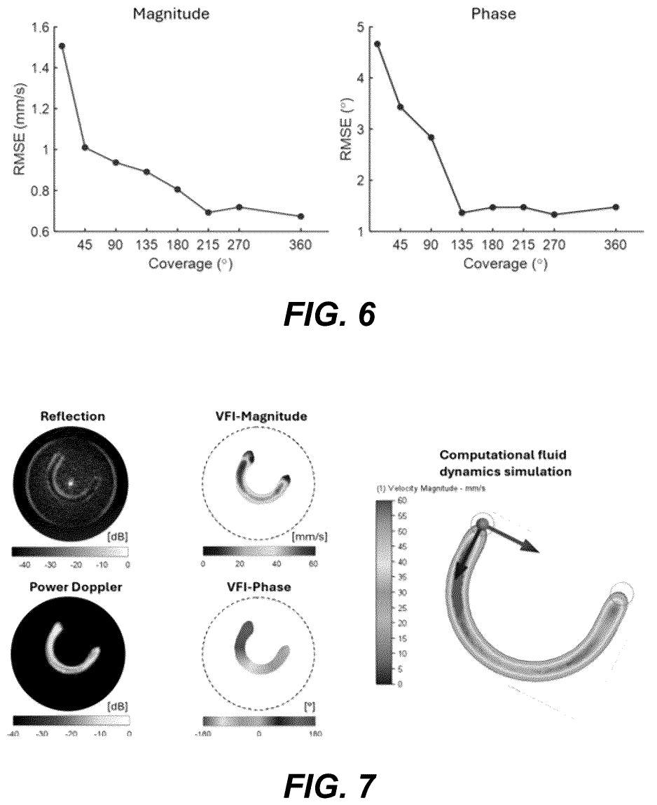

Further demonstration of the flexibility of using semi-enclosed array, the errors of estimated flow vectors are analyzed at different view coverage of the ring array (FIG. 6), using the same phantom as shown in FIGS. 4A-4F and FIGS. 5A-5F. The results show that phase error reduction is beneficial from the increasing view coverage around the object until no new angular information was obtained from the Doppler measurements. The fully enclosed ring array causes the redundancy in phase estimation while this redundancy is helpful to reduce the magnitude estimation errors. These results indicate using semi-enclosed array may also provide accurate phase estimation of vector flow while at the cost of slightly increased magnitude estimation errors.

Specifically, FIGS. 4A-4F compare the estimated magnitude maps generated using all views (FIG. 4B) and a single view (FIG. 4C) with the GT (FIG. 4A). The all-view reconstruction (FIG. 4B) closely resembles the GT, showing a more uniform magnitude distribution across the phantom, whereas the single-view reconstruction exhibits greater spatial fluctuation. The error maps (FIGS. 4D, 4E) further highlight this difference. The all-view error map (FIG. 4D) displays a centrally symmetric pattern with a root-mean-square error (RMSE) of 0.62 mm/s. In contrast, the RMSE increased to 1.22 mm/s along with higher spatial variance in the single-view reconstruction (FIG. 4E). For the all-view reconstruction, both the mean and STD of the angular deviation remained low across radial distances within 10 mm (FIG. 4F).

The accuracy of the estimated phase is of more interest for visualizing vector flow at video rates (>24 Hz), as the current phase map predicts the updated direction of the flow vectors displayed at the next time step. The errors in phase estimation were remarkably reduced in UST-VFI (FIGS. 5A-5C), with RMSE decreasing from 4.16° (FIG. 5E) to 0.93° (FIG. 5D). In contrast to the magnitude underestimation observed at the periphery (FIG. 4D), phase estimation was prone to greater errors near the center in both the all-view and single-view cases (FIGS. 5D & 5E), where velocity approaches zero. From the perspective of the autocorrelator, the estimated phase can only be zero if the signal remains unchanged across all frames over time. However, the stationary signals at the center were rejected along with the true clutter noise by the clutter filter. The remaining signals, primarily from electrical noise and motor vibrations, caused phase estimation errors. Nevertheless, our proposed UST-VFI outperformed the single-view VF reconstruction, achieving lower angular variations across a wide radial distance range in phase estimation (FIG. 5F).

In addition to the conventional reflection imaging capabilities, the system with ultrafast data acquisition rate can provide both functional and quantitative imaging modalities. FIG. 7 shows an example of vector flow imaging using wall-less flow phantom, demonstrating the feasibility of in-vivo blood flow imaging. The reconstructed magnitude of flow vectors is comparable to that of computational fluid dynamics simulation.

### Example 2: Methodology for Out-of-Plane Velocity Estimation Using Speckle Decorrelation

### Acquisition of Ultrasound Data for UST-VFI Processing

Ultrasound channel data and/or beamformed data using an enclosed or semi-enclosed ultrasonic transducer configuration is acquired as described in Example 1. A sequence of beamformed frames is generated at a selected frame rate (e.g., high-frame-rate imaging suitable for flow estimation). In some embodiments, complex beamformed data (e.g., IQ or analytic signal representations) are retained for subsequent processing.

Estimation of in-Plane Velocity Components Using the UST-VFI Method

UST-VFI processing is applied to the acquired data to estimate the blood flow velocity components within the imaging plane (e.g., lateral V_{z} and axial components Vₓ relative to the 2D acquisition geometry). The resulting in-plane velocity components may be produced as spatially resolved maps and may be time-resolved (e.g., per frame, per ensemble, or per temporal window), depending on the implementation.

### Definition of a Region of Interest for Decorrelation Analysis

A region of interest (ROI) corresponding to flow, such as a vessel lumen or other perfused region is identified and defined by using any suitable approach, including but not limited to: segmentation, thresholding, power Doppler information, prior anatomical knowledge, or user selection. Processing described below may be performed for all spatial locations within the ROI and optionally omitted outside the ROI.

### Computing Temporal Correlation as an Exponential Function of Time Aag

For each spatial location within the ROI (e.g., each pixel or each patch center), a local spatial neighborhood (“patch”) is defined comprising a plurality of intensity samples. For a selected set of temporal lags (e.g., several frame separations), a correlation value between intensity patches extracted from temporally separated frames is computed. For a given temporal lag n, normalized correlation over multiple frame pairs K separated by that lag and average the resulting correlation values to reduce stochastic variability is determined, thereby producing an estimated correlation function R(n) as a function of frame lag:

$$R ( n ) = \frac{1}{K} {\sum }_{i = 1}^{K} n o r m x c o rr ( {I}_{i} , {I}_{i + n} ) \qquad ( 6 )$$

In some embodiments, the temporal lag range, patch size, patch overlap, and correlation computation may be selected to balance noise robustness, spatial resolution, and computational complexity.
Estimation of a Decorrelation Value from the Correlation Function

For each spatial location (or patch center), determine a decorrelation value τ that characterizes the decay of the intensity correlation function R(n) with increasing temporal lag. In some embodiments, the decorrelation value is obtained by fitting a parametric model to the measured correlation function (e.g., a Gaussian decay model):

$$R ( n ) = {e}^{- \frac{{n}^{2}}{2 P R {F}^{2}} τ} \qquad ( 7 )$$

In other embodiments, the decorrelation value may be extracted using non-parametric measures or alternative curve-fitting models. The resulting decorrelation value may be computed per spatial location and optionally per temporal window (e.g., for pulsatile flow.)

### Relating Decorrelation Value to Magnitude of 3D Motion

The decorrelation value is related to scatterer displacement and therefore depends on the magnitude of the underlying local velocity vector, including contributions from in-plane and out-of-plane motion. In some embodiments, this relationship incorporates spatial correlation parameters corresponding to effective correlation widths of the ultrasound system and beamformed point spread function along different spatial directions (e.g., within-plane and elevational directions). The spatial correlation parameters may be predefined, calibrated, or estimated from data, and may be treated as constant or spatially varying in different implementations.

Estimating Out-of-Plane Velocity Component Using Known in-Plane Components

Using the decorrelation value t estimated for a location and the in-plane velocity components produced by the UST-VFI processing (Vₓ and V_{z}), compute an estimate of the out-of-plane velocity component that is consistent with the decorrelation relationship:

$$τ = ( \frac{{V}_{x}^{2}}{{σ}_{x}^{2}} + \frac{{V}_{y}^{2}}{{σ}_{y}^{2}} + \frac{{V}_{z}^{2}}{{σ}_{z}^{2}} ) \qquad ( 8 )$$

$$\vert {V}_{y} \vert = \sqrt{τ - \frac{{V}_{x}^{2}}{{σ}_{x}^{2}} - \frac{{V}_{z}^{2}}{{σ}_{z}^{2}}} \qquad ( 9 )$$

In some embodiments, the out-of-plane component is obtained by solving an expression in which the decorrelation-derived motion magnitude is decomposed into contributions from in-plane and out-of-plane components. In some embodiments, non-physical solutions (e.g., negative values under a square root) are handled using constraints or clipping operations.
Output of a Three-Dimensional Velocity Estimate and/or Derived Flow Metrics

Combine the estimated in-plane velocity components and the estimated magnitude of the out-of-plane velocity component obtained from the speckle decorrelation analysis to form a three-dimensional blood flow velocity representation. In some embodiments, the sign or direction of the out-of-plane velocity component is determined using additional information, including but not limited to: (1) projection of the in-plane velocity components onto a known or assumed three-dimensional flow direction, for example based on anatomical or physiological context such as an expected direction of blood flow along a vessel from a proximal region toward a distal region (e.g., from the heart toward downstream vasculature); continuity or other fluid-dynamics-based constraints; (3) anatomical or geometric constraints associated with the vessel or flow region; and/or (4) prior knowledge of expected flow direction.

The resulting three-dimensional velocity field may be output as a per-pixel or per-patch spatial map and may be time-resolved. In some embodiments, derived metrics such as volumetric flow, flow rate, velocity magnitude, flow direction, or combinations thereof may additionally be computed from the three-dimensional velocity field.

The steps of this example may be implemented in a variety of system configurations and processing pipelines. Non-limiting variations include: selection of ultrasonics transducers, selection of patch size and overlap; selection of temporal lag range; use of sliding windows for time-varying flow; alternative correlation estimators; alternative decorrelation models; and acceleration using parallel hardware (e.g., GPU) or dedicated signal-processing hardware.

### Example 3: Out-of-Plane Velocity Estimation Using Speckle Decorrelation

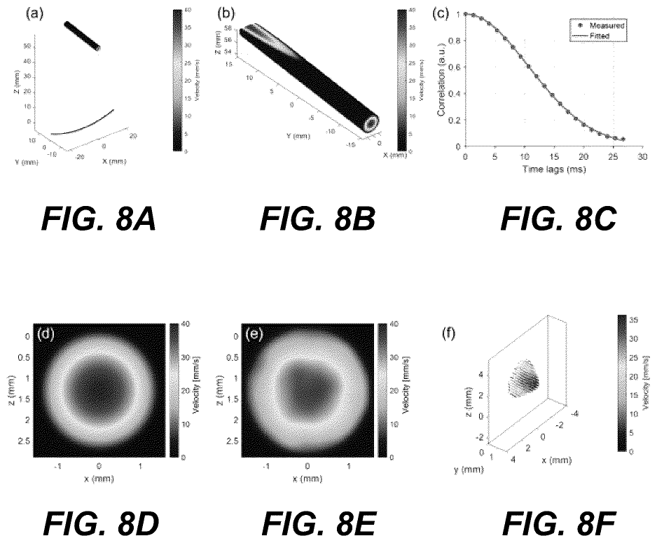

The speckle decorrelation-based out-of-plane velocity estimation method was validated using numerical simulations performed with the Field II ultrasound simulation platform. A straight cylindrical tube having a length of 30 mm and a radius of 1.5 mm was positioned at the center of the imaging probe. The tube axis was tilted by approximately 5 degrees relative to the imaging plane, as illustrated in FIG. 8A. A laminar flow condition was simulated within the tube, resulting in a spatially varying velocity distribution representative of blood flow under laminar conditions, as shown in FIG. 8B. Ultrasound data were generated using a tomographic scanning configuration in which the transducer probe was rotated to eight distinct angular views around the flow phantom. The acquisition rate was set to approximately 6 kHz, resulting in an effective frame rate of approximately 750 Hz after beamforming. Scatterers within the tube were displaced at each time step of the data acquisition according to the prescribed laminar flow velocity distribution. A total of 112 beamformed frames were generated and used for the speckle decorrelation analysis.

The speckle decorrelation values were estimated by computing temporal intensity correlation and fitting the resulting correlation decay curves using a Gaussian model, as illustrated in FIG. 8C. Using the estimated decorrelation values in combination with the in-plane velocity components obtained from the tomographic vector flow imaging method disclosed in the prior application, the magnitude of the out-of-plane velocity component was reconstructed. The reconstructed out-of-plane velocity distribution was compared with the ground-truth velocity profile obtained from the simulation, as shown in FIGS. 8D and 8E. The results demonstrate that the speckle decorrelation-based method successfully recovered the laminar out-of-plane velocity profile, with a root-mean-square error of approximately 3.49 mm/s. By combining the in-plane velocity components obtained using the previously disclosed UST-VFI method with the out-of-plane velocity estimated via speckle decorrelation, a three-dimensional vector flow representation was obtained. These simulation results demonstrate the feasibility and accuracy of the disclosed approach for extending two-dimensional tomographic vector flow imaging to three-dimensional blood flow measurement.

While various embodiments have been described above, it should be understood that such disclosures have been presented by way of example only and are not limiting. Thus, the breadth and scope of the subject compositions and methods should not be limited by any of the above-described exemplary embodiments but should be defined only in accordance with the following claims and their equivalents.

The above description is for the purpose of teaching the person of ordinary skill in the art how to practice the present application, and it is not intended to detail all those obvious modifications and variations of it which will become apparent to the skilled worker upon reading the description. It is intended, however, that all such obvious modifications and variations be included within the scope of the present application, which is defined by the following claims. The claims are intended to cover the components and steps in any sequence which is effective to meet the objectives they intended, unless the context specifically indicates the contrary.

What is claimed is:

1. A method of ultrasound tomography angle-independent vector blood flow measurement, comprising the steps of:
(1) acquiring data using an ultrasound transducer array with extended aperture size, wherein the extended aperture size is reflected in a relative ratio between the array aperture size and a largest dimension of an image object, wherein diverging waves are transmitted by a portion of the array using one or more elements of the array, wherein a divergent wavefront provides a field-of-view comprising anisotropic Doppler interrogation angles, wherein, after transmission of the diverging waves, the extended aperture of the array captures at least a majority of echo reflection waves,
wherein the data is acquired by (a) rotating active views around the image object, wherein signal transmissions and echo receptions are repeated at each active view position, wherein a Doppler shift is acquired from multiple directions; and (b) distributing tomographic scanning sparsely and repeating at an ultrafast pulse repetition rate (PRF) (kHZ), wherein in one round of tomographic scanning the date acquired from a total number of M views are collected and the achievable frame rate is PRF/M;
(2) dividing the portion of the ring array receiving the echoes at each view into N sub-apertures, wherein the echoes from each sub-aperture are beamformed in parallel at each time point, and a slow-time relative to the PRF ensemble of beamformed reflection images are saved, and estimating color Doppler shifts over the ensembled images;
(3) collecting total MN Doppler measurements with tomographic scanning and sub-aperture beamforming techniques;
(4) constructing a forward matrix A, wherein for the color Doppler measurements only the frequency shifts along the wave propagation direction are measured to produce a measurement matrix B, wherein a transmitted Doppler direction is pointing from pixels to the virtual point source of the diverging waves, and wherein a received Doppler direction is pointing from pixels to the virtual received source at the center of each sub-aperture;
(5) reconstructing, after completion of the forward matrix A and measurement matrix B, the axial and lateral components of flow vectors by a least-squares solver; and
(6) displaying an image for the image object.

2. The method of claim 1, wherein the ultrasound transducer array is an enclosed ring array.

3. The method of claim 1, wherein the ultrasound transducer array is a partially enclosed ring array.

4. The method of claim 1, wherein the ultrasound transducer array is a single continuously curved transducer array.

5. The method of claim 1, wherein the ultrasound transducer array comprises two or more curved subarrays.

6. The method of claim 1, wherein the active views are rotated around the imaged object electronically.

7. The method of claim 1, wherein the active views are rotated around the imaged object mechanically.

8. The method of claim 1, wherein the Doppler shift is acquired in a lateral direction relative to the image object.

9. The method of claim 1, wherein the sparsity and spatial distribution of tomographic views changes to accommodate different coverage and shape of the transducer array.

10. The method of claim 1, wherein clutter filtering is employed on the ensemble of reflection images after beamforming.

11. The method of claim 1, wherein clutter filtering is employed on the ensemble of reflection images before beamforming.

12. The method of claim 1, wherein estimation of color Doppler shifts uses a lag-one autocorrelator in a sliding window.

13. The method of claim 1, wherein the image is a power Doppler imaging of the image object.

14. The method of claim 1, wherein the image is vector flow imaging of the image object.

15. A method for three-dimensional (3D) ultrasound tomography vector flow imaging (3D UST-VFI), comprising the steps of:
(1) acquiring ultrasound data for UST-VFI processing;
(2) estimating in-plane velocity components using the method of claim 1;
(3) defining a region of interest for decorrelation analysis;
(4) computing temporal correlation as an exponential function of time lag;
(5) estimate a decorrelation value from the correlation function;
(6) relating the decorrelation value to magnitudes of 3D motion;
(7) estimate the out-of-plane velocity component using the known in-plane components; and
(8) output a three-dimensional velocity estimate and/or derived flow metrics.

16. A system for ultrasound tomography angle-independent vector blood flow measurement, comprising:
(1) an ultrasound transducer array with extended aperture size, wherein the extended aperture size is reflected in a relative ratio between the array aperture size and a largest dimension of an imaged object, wherein diverging waves are transmitted by a portion of the array using one or more elements of the array, wherein a divergent wavefront provides a field-of-view comprising anisotropic Doppler interrogation angles, wherein, after transmission of the diverging waves, the extended aperture of the ring array captures at least a majority of echo reflection waves;
(2) a rotational mechanism connected to the ultrasound transducer array, wherein the rotational mechanism rotates the ultrasound transducer array through active views around the imaged object;
(3) a computer processor, wherein the computer processor (a) estimates color Doppler shifts over the ensembled images, (b) constructs a forward matrix A, wherein for the color Doppler measurements only the frequency shifts along the wave propagation direction are measured to produce a measurement matrix B, wherein a transmitted Doppler direction is pointing from pixels to the virtual point source of the diverging waves, and wherein a received Doppler direction is pointing from pixels to the virtual received source at the center of each sub-aperture, and (c) reconstructs, after completion of a forward matrix A and measurement matrix B, the axial and lateral components of flow vectors by a least-squares solver; and
(4) a display device connected to the computer processor.

17. The system of claim 16, wherein the ultrasound transducer array is an enclosed ring array.

18. The system of claim 16, wherein the ultrasound transducer array is a partially enclosed ring array.

19. The system of claim 16, wherein at each active view, a single transducer element transmits a diverging wavefield propagating through the image object.

20. The system of claim 16, wherein at each active view, multiple transducer elements transmit a diverging wavefield propagating through the image object.
